(19) United States
(12) Patent Application Publication
(10) Pub. No.: US 2008/0169307 A1
(43) Pub. Date: Jul. 17, 2008

(54) E-Module with Interface-Proof Inductive Sensing

(76) Inventor: Michael Hofstetter, Zollikofen (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/674,519

(22) Filed: Feb. 13, 2007

(30) Foreign Application Priority Data
Feb. 14, 2006 (DE) 10 2006 006 784.3

(51) Int. Cl. B67D 5/30

(52) U.S. Cl. 222/14

(57) ABSTRACT

A dose setting module and method for use with an injection device, the module including at least one sensor element and at least one sensor actuating element, wherein the at least one sensor element and at least one sensor actuating element at least partially overlap along the longitudinal axis of the injection device and can be moved into at least one position in which they at least partially overlap in the circumferential direction of the injection device, and wherein the at least one sensor element generates a magnetic field and, in the at least one position, the at least one sensor actuating element causes a change in the magnetic field.

### CROSS-REFERENCE TO RELATED APPLICATION

This application claims priority to German Application No. 10 2006 006 784.3, filed on Feb. 14, 2006, the entire content of which is incorporated herein by reference.

### BACKGROUND

The present invention relates to devices for delivering, administering, injecting, infusing or dispensing substances, and to methods of making and using such devices. More particularly, it relates to setting, selecting and/or monitoring an amount or dose of a substance to be delivered, administered, injected or dispensed, to a dose-setting module, and to a method of determining a dose setting for an infusion or injection device. More particularly, the present invention relates to a device and a method of administering a liquid in set doses, e.g., for medical applications or uses, wherein the set dose of liquid dispensed or to be dispensed can be detected contactlessly by inductive sensors.

To enable substances and/or liquids, such as insulin, hormone preparations, etc., to be dispensed from a medical instrument such as an injection device or an injection pen in a selected amount or dose, and for the amount or does to be measured as exactly as possible, the selected or set dose and the procedure of setting the dose should be able to be accurately set, detected, monitored and/or assessed. Typically, if an incorrect dose is set or if there is another discrepancy regarding a dose, a warning signal may be emitted or displayed or administration or delivery of the dose is prevented.

Generally, the dose to be administered from an injection device, e.g. an injection pen, is set by turning a dose setting knob and delivered by then depressing the dose setting knob or another trigger structure. Known devices are based on mechanical principles and may, therefore, be complex to produce and relatively inaccurate. Due to their construction, it may also be that mechanical systems are not sufficiently sealed, thereby allowing penetration by air or dust which can reduce the accuracy and service life of the systems.

Patent specification DE 101 33 216 discloses a device for administering set doses of a liquid, in particular for medical applications, whereby the set dose of the liquid dispensed or to be dispensed is determined on the basis of the relative position of a rotor with respect to a stator. To detect the position of the rotor relative to the stator at least two electrodes are disposed on the rotor and at least two electrodes are disposed on the stator so that the electrodes partially overlap to form a capacitor in at least one rotational position of the rotor relative to the stator. However, capacitive sensors react to all materials, irrespective of whether they are electrically conductive or have insulating properties. The sensors may, therefore, detect metal or plastic and are susceptible to faults due to dirt.

### SUMMARY

One object of the present invention is to overcome disadvantages known from the prior art. Another object of the present invention is to provide an infusion or injection device including a device for setting or selecting a dose, and a device and method of detecting and/or monitoring a set or selected dose setting which enables the dose setting to be detected easily and exactly.

In one embodiment, the present invention comprises a dose setting module and method for use with an injection device, the module including at least one sensor element and at least one sensor actuating element, wherein the at least one sensor element and at least one sensor actuating element at least partially overlap along the longitudinal axis of the injection device and can be moved into at least one position in which they at least partially overlap in the circumferential direction of the injection device, and wherein the at least one sensor element generates a magnetic field and, in the at least one position, the at least one sensor actuating element causes a change in the magnetic field.

In one embodiment, a dose setting device or module for an infusion and/or injection device in accordance with the present invention has a number of sensor elements, e.g., 1-5 or more such elements, which may be spaced apart from one another along the longitudinal axis or in the longitudinal direction of the injection device. The sensor elements may be disposed inside the injection device, they may be disposed on the dose setting mechanism of the device, on a threaded rod or a rotor of the device, or, alternatively, opposite the dose setting mechanism, for example on a circuit board. The dose setting module also has a number of sensor actuating elements, e.g., 1-5 or more such elements, which can cause a change in a magnetic field. The sensor actuating elements may take the form of rotatable elements or cams that may be disposed offset from one another along the longitudinal axis of the injection device, for example on a rod. The sensor actuating elements may be disposed inside the injection device or on the dose setting mechanism. In some embodiments, they may take the form of switching rings made from metal which surround the dose setting mechanism or threaded rod. The switching rings may be formed in an annular or circular arrangement around the dose setting mechanism or threaded rod and may be provided with cams as sensor actuating elements. In some preferred embodiments, at least two of the cams have a relatively different length and/or have a relatively different shape, such as rectangular, triangular, circular, semi-circular, or other suitable shape.

In some embodiments, at least one of the sensor actuating elements is respectively offset from the others along the longitudinal axis or in the longitudinal direction of the injection device and is shaped or designed so that it is able to interfere with, change or have an effect on the magnetic field of an oppositely lying sensor element. At one position in the longitudinal direction of the injection device, it is also possible to provide more than one sensor actuating element, for example along or about the circumference of the dose setting module or threaded rod associated with the injection device or dose setting module.

In some embodiments, the sensor actuating elements may be disposed circumferentially about the dose setting module, which, in some embodiments, is circular or annular in shape. In some embodiments, they may be disposed circumferentially about a circular or annular rod or threaded rod associated with the injection device or dose setting module. In some embodiments, the sensor actuating elements are spaced equally or constantly with respect to one another about the circumference and are in the same position along the longitudinal axis, offset from one another in the circumferential direction by 90°, 180° and/or 270°. The sensor actuator elements may also be spaced apart from one another circumferentially and respectively by 45°. In some preferred embodiments, the sensor elements and the sensor actuating elements may be disposed so that a sensor element and a sensor actuating element at least partially overlap along the longitudinal axis of the injection device, and/or they may be disposed lying opposite one another.

In some embodiments of the present invention, if the sensor actuating elements are disposed opposite the dose setting mechanism of the injection device, for example, the sensor elements may be disposed or located on the dose setting mechanism. The sensor elements may also be mounted opposite the dose setting mechanism, in which case the sensor actuating elements may be provided on the dose setting mechanism in the form of switching rings, for example, and disposed along the circumference of the dose setting mechanism or threaded rod, for example. If the sensors are disposed opposite the dose setting mechanism, for example, and the sensor actuating elements are disposed on the dose setting mechanism or threaded rod, the position of the sensor actuating elements can be changed in the circumferential direction of the threaded rod or the infusion or injection device by rotating the threaded rod. Accordingly, a sensor actuating element can be moved by a movement of the dose setting mechanism, such as a rotation or sliding motion of the dose setting mechanism, by rotating or sliding, so that the sensor actuating element and the sensor at least partially, but in some cases totally, overlap in the circumferential direction of the infusion or injection device. The sensor actuating element or the threaded rod may also latch in this position, thereby indicating to the user that a desired or specific dose or partial dose has been reached or set. In some preferred embodiments, the dose setting module has at least one sensor actuating element which changes or interferes with a magnetic field generated by a sensor element when moved into a position in which it at least partially or totally overlaps with the sensor element. Different types of sensor actuating elements may also be disposed on the dose setting mechanism or on the switching ring which are not able to screen, weaken, interfere with or change the field, when they lie opposite a sensor element or overlap one, or are able to do so to only a slight degree, for example by a quarter or a half, or are able to do so to a high degree, for example up to three quarters or completely. As a result of this screening, weakening, interference or change, an electronic evaluation system is able to draw conclusions about the position or rotational position of the sensor actuating elements, and hence about the dose setting mechanism or threaded rod, thereby making it possible to ascertain how much of the liquid contained in the injection device can or should be dispensed. In some preferred embodiments, a change in a magnetic field of a sensor element is recognized or detected by the sensor element itself. In particular, when a change in the magnetic field is detected, an evaluation unit (which may take the form of or comprise a suitable microprocessor, computer, etc.) which is connected to the sensor elements emits a warning signal (the evaluation unit may generate the signal or cause it to be emitted). If there is no change in the magnetic field, the evaluation unit may emit a first signal for the co-operating sensor element, for example a low signal, for example a logical “0”, and when a change is detected in the magnetic field, a second signal different from the first signal is emitted for this sensor element, such as a high signal or a logical “1”.

In some embodiments, a dose setting module in accordance with the present invention may have a reference sensor element, which is able to recognize or detect an external magnetic field. The reference sensor element may be spaced apart from the other sensor elements of the infusion or injection device and is separated from the sensor elements by housing parts, for example. The reference sensor element may be disposed on or opposite the dose setting mechanism of the infusion or injection device so that it does not overlap with any of the sensor actuating elements disposed on the dose setting mechanism in the longitudinal direction of the infusion or injection device and is therefore not susceptible to interference.

In some preferred embodiments, the sensor element comprises a coil and a magnetic core, such as a ferrite core, and a current is able to flow through the coil, thereby enabling a concentric magnetic field to be created at the sensor element, for example. The current, and hence the magnetic field, may be kept constant, in which case the intensity may be pre-defined and known to the evaluation unit, thereby enabling a constant magnetic field or a magnetic field of constant intensity to be generated. A screen, such as an electrically conducting housing shell, may be mounted around the injection or infusion device or around the dose setting module, which is able to screen the sensor elements and the sensor actuating systems or sensor actuating elements from the external magnetic field, to prevent changes in the magnetic fields of the sensor elements, for example.

The sensor actuating elements of each sensor actuating system may be of different designs or shapes or they may be of different lengths. For example, the sensor actuating elements may be of such a length that they are not able to change or interfere with the magnetic field of a sensor when the sensor is lying opposite. The sensor actuating elements may also be of a length or shape such that they almost completely disrupt the magnetic field of an oppositely lying sensor. In some embodiments, the sensor actuating elements may also be of a shape or length such that they change or interfere with the magnetic field of a sensor element up to or in excess or one or more pre-set critical values.

Different dose settings can be recognised and detected by means of the dose setting module in accordance with the present invention. For example, a dose to be dispensed can be set by the dose setting or rotating knob and the quantity of the dose to be dispensed may depend on the rotational position or the rotated angle of the dose setting knob. For example, depending on the rotational position or the rotation angle, the dose setting mechanism can be closed at the set dose.

In some preferred embodiments, the dose setting mechanism can be latched in pre-defined positions or at predefined rotation angles, such as 45°, 90°, 135°, 180° and other multiples of 45° or multiples of 90°, for example. The sensor actuating elements and/or the sensors may be located, disposed or arranged so that the rotational position or the rotation angle of the dose setting mechanism is clearly determined or assigned to a quantity or dose to be dispensed. For example, the dose setting module may have a sensor and two sensor actuating elements or cams at the same position in the longitudinal direction of the injection device, in which case they may be offset from one another by 180° in the circumferential direction. One sensor element may be designed so that the magnetic field of the sensor element is not disrupted when the sensor actuating element is lying opposite the sensor element and the other sensor actuating element may be designed so that the magnetic field of the sensor element is disrupted when the sensor actuating element is lying opposite the sensor element. The sensor actuating elements may be offset from one another by 180°, for example, and when a disruption to the magnetic field is recognized or detected, a logical “1” is output and if there is no change in the magnetic field a logical “0” is output by an evaluation unit, so that the positions or dose settings may differ from one another.

In some embodiments, finer dose settings or dispensing quantities can be set for the product to be dispensed, in which case more than two, for example four, sensor actuating elements or cams are provided on the dose setting mechanism, and one or two or more sensors are provided. For example, all four sensor actuating elements may differ in design and may lie in the same position as the sensor element in the longitudinal direction of the injection device so that the sensor actuating elements change the magnetic field of the sensor to differing degrees when they are lying opposite it. Alternatively, two cams may be disposed respectively in the same longitudinal position as a first sensor and two other cams may be disposed in a same longitudinal position of the injection device as a second sensor, in which case the sensor actuating elements may be disposed offset from one another in the circumferential direction of the dose setting mechanism, and are offset from one another by 90° respectively. This being the case, four different rotational positions or setting positions of the rod or the dose setting module can be clearly detected, specifically offset from one another by 90°. Alternatively, two cams may be disposed offset from one another respectively in the longitudinal direction of the injection device, in which case the two cams may be of different designs so that one cam is able to disrupt the magnetic field of an oppositely lying sensor, whilst the other cam causes a disruption when it is lying opposite a sensor element. Accordingly, the cams are offset from one another by 45° in the circumferential direction, thereby enabling eight dose settings to be detected.

In some embodiments, the dose setting module may also have a resetting mechanism or locking mechanism, such as a reset switching ring, which may be disposed opposite the dose setting mechanism of the infusion or injection device, on the dose setting mechanism or in another suitable location, e.g., in the form of a switching ring disposed around the threaded rod. The resetting mechanism at least partially but, in some preferred embodiments fully, overlaps a sensor element in the longitudinal direction of the injection device. The resetting mechanism may be moved by turning the dose setting mechanism for example, so that a sensor element and the re-setting mechanism at least partially or totally overlap in the circumferential direction of the dose setting mechanism or infusion or injection device. As a result, the magnetic field of the sensor element is at least partially or totally changed by the resetting mechanism.

In some embodiments, the locking mechanism of the injection device may have a switching ring or slide which can be displaced relative to a housing of the injection device in the radial direction with respect to the longitudinal axis of the injection device. To this end, the slide may be provided in the form of an oval ring disposed around a sleeve or the dose setting mechanism. The slide may sit in a released position in which it lies opposite the surface of the sleeve or dose setting mechanism.

In some embodiments, the slide may have a locked position in which the dose setting knob is pushed into the housing, causing the sleeve to be pushed forward in the longitudinal direction of the injection device, for example, until a projection of the slide pointing in the direction of the sleeve locates in or moves into a groove on the sleeve, thereby preventing the dose setting knob from being pushed in farther. The slide may be disposed opposite a sensor element. In the released position, a first distance can be defined between the sensor element and the surface of the slide or switching ring facing the sensor element. When the slide is in the locked position, the projection may locate in the groove, thereby enabling the distance of the surface of the slide from the laser detector to be made longer. This change of distance may be recorded by the sensor element and forwarded as a measurement signal to a microprocessor, which can then output the locked position on a display of the injection device.

In accordance with the present invention, in one embodiment of a method for detecting a dose setting of an infusion or injection device, a sensor element which may be disposed on a dose setting mechanism, on a sleeve or threaded rod of the injection device or opposite a sleeve of the injection device generates a magnetic field, which is, in some preferred embodiments, concentric. This magnetic field may be disrupted by means of a sensor actuating element which may be disposed on or opposite the dose setting mechanism of the injection device for example. Alternatively, one or more sensor elements may be disposed opposite the dose setting mechanism of the injection device and one or more sensor actuating elements may be disposed along the threaded rod or dose setting mechanism of the injection device. The magnetic field of the sensor element or elements is initially not disrupted if an external magnetic field is not present and if none of the sensor actuating elements is interfering with the magnetic field. When the dose setting mechanism is turned, for example to set a desired dose, one or more sensor actuating elements may overlap with the sensor element in the circumferential direction of the injection device, so that the magnetic field of the sensor element is disrupted.

In some embodiments, one or more sensor actuating elements may be provided on the dose setting mechanism or in the injection device, which are able to overlap with the sensor element in the circumferential direction of the dose setting mechanism or injection device when the dose setting mechanism is turned. Some sensor actuating elements may be of a shape or design which is such that in the overlapping or oppositely lying position, no change in field occurs, a slight change in field occurs or a virtually full change occurs.

For example, if an external magnetic field exists in the area around the injection device, such as a magnetic interference field, the external magnetic field can be detected by a reference sensor element. An external magnetic field can also be detected by the sensor elements. If sensor actuating elements are disposed so that it is never possible for all of the magnetic fields to be disrupted by sensor actuating elements in every position of the dose setting mechanism, it can be concluded from a change in all of the magnetic fields of the sensor elements that an external magnetic field is present.

If a sensor element detects that the magnetic field generated by the sensor element has been changed, a first signal such as a high signal or a logical “1” can be output for this sensor element by an evaluation unit. In the event of an unchanged magnetic field, on the other hand, the evaluation unit can output a second signal such as a low signal or a logical “0” that is different from the first signal. The sensor element can therefore be operated as a “binary sensor”. In some preferred embodiments, the sensor actuating elements are disposed such that, in no rotational position of the dose setting mechanism is it possible for the magnetic fields of all the sensor elements to be changed, which means that a logical “1 can not be output by the evaluation unit for each sensor element in any rotational position. When an external magnetic field is applied or exists, the magnetic fields of all the sensor elements can be changed so that a logical “1” can be output for every sensor element and a presence of an external magnetic field can be detected.

If an external magnetic field is recognized or detected, the evaluation unit is able to regulate the sensor elements so that the external magnetic field is compensated at the sensor elements. For example, the evaluation unit is able to send a signal to the sensor elements, specifying the degree to which the current passing through the coil of the sensor element must be changed or increased or reduced, or the degree to which the magnetic field of the sensor element must be changed, increased or reduced to compensate for the external magnetic field. The evaluation unit may also compensate for the external magnetic field by making allowance for the external magnetic field or the intensity of the external magnetic field in the calculations that are run. For example, a sensor element is able to detect that its magnetic field has changed. An evaluation unit can specifically determine the degree to which the magnetic field has changed. Measuring devices such as Hall-effect sensors may be connected to the sensor element and/or the evaluation unit, for example, which are able to measure the flux density or the magnetic field intensity of the magnetic field. The unchanged field intensity or the unchanged flux density of every magnetic field is known, thereby enabling conclusions to be drawn about the intensity of the change to the changed field and hence about the sensor actuating element or the external magnetic field inducing the change on the basis of the measurement of the changed field. This change can be compensated or calculated out during the subsequent calculations. The evaluation unit may also change the current through the coil so that the change or the external magnetic field is compensated and the magnetic field outwardly possesses the same field intensity that it would if the magnetic field were not being subjected to a change.

In some embodiments, in the evaluation unit the degree to which the magnetic field has been changed or altered by a sensor actuating element can be determined, thereby enabling conclusions to be drawn about the sensor actuating element inducing the change and about the dose setting. For example, a critical value for a change may be pre-set in the evaluation unit, and when it is exceeded, it may be assumed that the magnetic field was changed, in which case a first signal such as a high signal or a logical “1” is output by the evaluation unit. If, on the other hand, there is a drop below the pre-set critical value, it may be assumed that no change has occurred in the magnetic field due to a sensor element and the evaluation unit can output a second signal such as a low signal or a logical “0” different form the first signal.

In some embodiments, another critical value may be pre-set in the evaluation unit and when it is exceeded, it is assumed that an external magnetic field is present. This external magnetic field may be detected by the reference sensor element. The critical value may also be set so that when exceeded, it is assumed that the external magnetic field is so intense that it can no longer be compensated by the evaluation unit and the evaluation unit must output an error signal.

### BRIEF DESCRIPTION OF THE DRAWINGS

FIG. 1 illustrates a structure of an inductive sensor;

FIG. 2 illustrates an embodiment of a dose setting module in accordance with the present invention; and

FIG. 3 illustrates an exemplary embodiment of an injection device with an embodiment of a dose setting module in accordance with the present invention.

### DETAILED DESCRIPTION

With regard to fastening, mounting, attaching or connecting the components of the present invention, unless specifically described as otherwise, conventional mechanical fasteners and methods may be used. Other appropriate fastening or attachment methods include adhesives, welding and soldering, the latter particularly with regard to the electrical features of the invention. In embodiments with electrical features or components, suitable electrical components, e.g. circuitry, wires, chips, boards, microprocessors, processors, computers, sensors, inputs, outputs, displays, control components, etc., may be used. Generally, unless otherwise indicated, the materials for making the present invention and/or its components may be selected from appropriate materials such as metal, metallic alloys, ceramics, plastics, etc.

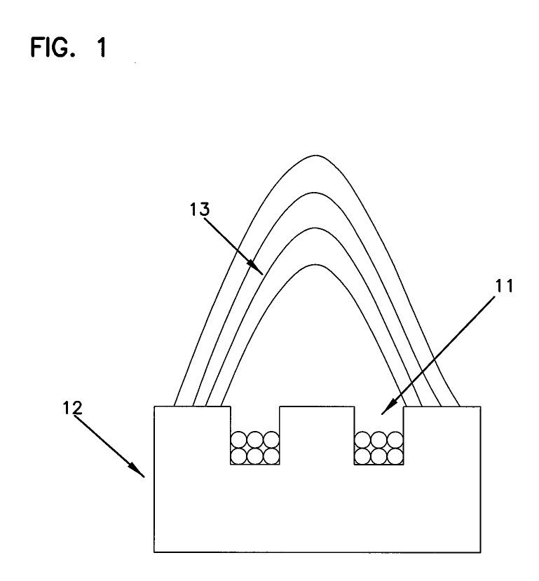

FIG. 1 illustrates an inductive sensor with a coil 11 capable of conducting current and a ferrite core 12 inserted in a dose setting module or an injection device in accordance with the present invention. A magnetic field 13 can therefore be generated when current is flowing through the coil 11. As a result of the power flow through the coil, a concentric magnetic field is created, which may extend in a semi-circular or semi-elliptical arrangement on the top face of the sensor element and which may also extend through the ferrite core or ferromagnetic core. The sensor may specifically be provided with a measuring device for measuring the current flow through the coil, the flux density of the magnetic field or field intensity of the magnetic field, such as a Hall-effect sensor, or the sensor may be connected to the latter. The magnetic flux density of the magnetic field can be determined by measuring a voltage, such as a Hall-effect voltage for example, which can occur in the conductor or coil due to a separation of the charge in the conductor or coil, and the flux density may be proportional to the measured voltage. If a voltage change is measured or detected by the measuring device, the intensity of the voltage change or the intensity of the change in the magnetic flux density can be determined in the evaluation unit, thereby also enabling conclusions to be drawn about the degree of change caused.

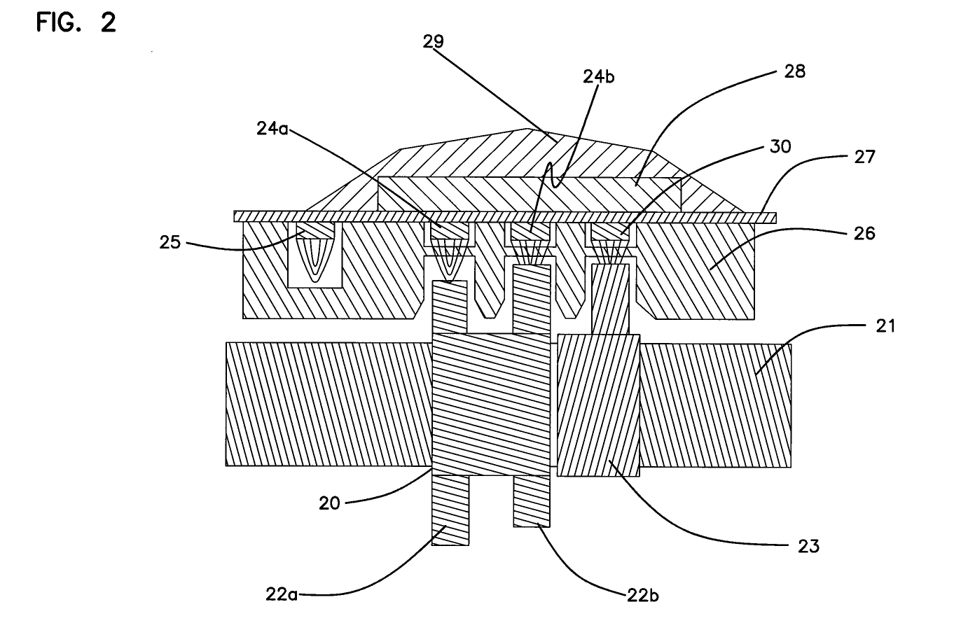

FIG. 2 illustrates an embodiment of a dose setting module in accordance with the present invention in an injection device with a dose setting mechanism 21, on which a switching ring 20 of metal and a reset switching ring 23 of metal are disposed. FIG. 2 also illustrates sensors 24a, 24b, the design of which may be the same as that of the sensor illustrated in FIG. 1, a reference sensor 25 and a module base 26, which are mounted on a circuit board 27. Also provided on the circuit board 27 are a chip 28 and a globe-top 29 serving as a seal or cover above the chip 28. A magnetic field can be generated by the sensors 24a, 24b. In the position illustrated in FIG. 2, the left-hand sensor actuating element 22a of the switching ring 20 is disposed so that it lies opposite the left-hand sensor 24a in the circumferential direction of the dose setting mechanism 21 and also lies opposite the left-hand sensor 24a in the longitudinal direction of the dose setting mechanism 21. The left-hand sensor actuating element 22a is of such a shape or length that it does not change or interfere with the magnetic field of the left-hand sensor 24a at all or does so only slightly. The right-hand sensor actuating element 22b of the switching ring 20 is of such a design or length that, as illustrated in FIG. 1, it changes or interferes with the magnetic field of the middle sensor 24b when overlapping the middle sensor 24b in the longitudinal direction and in the circumferential direction of the dose setting mechanism 21. This change may be recognised or detected by the chip 28, for example, and the chip 28 may emit a pre-defined signal for the middle sensor 24b, such as a high signal. If the dose setting mechanism 21 were turned about the longitudinal axis of the dose setting mechanism or injection device by a further 180°, for example, the left-hand sensor actuating element 22a would change or interfere with the magnetic field of the left-hand sensor 24a, whilst the magnetic field of the middle sensor 24b would remain unchanged and there would no longer be any change or interference from the right-hand sensor actuating element 22b. In this case, the chip 28 could emit a high signal for the left-hand sensor 24a and a low signal for the middle sensor 24b. The presence of an external magnetic field, such as an interference field, may be detected by means of a reference sensor 25. If an external magnetic field is detected, allowance may be made for it when evaluating the changes to the magnetic fields of the sensors 24a, 24b or the magnetic fields of the sensor elements 24a, 24b may be changed so that the external magnetic field is compensated.

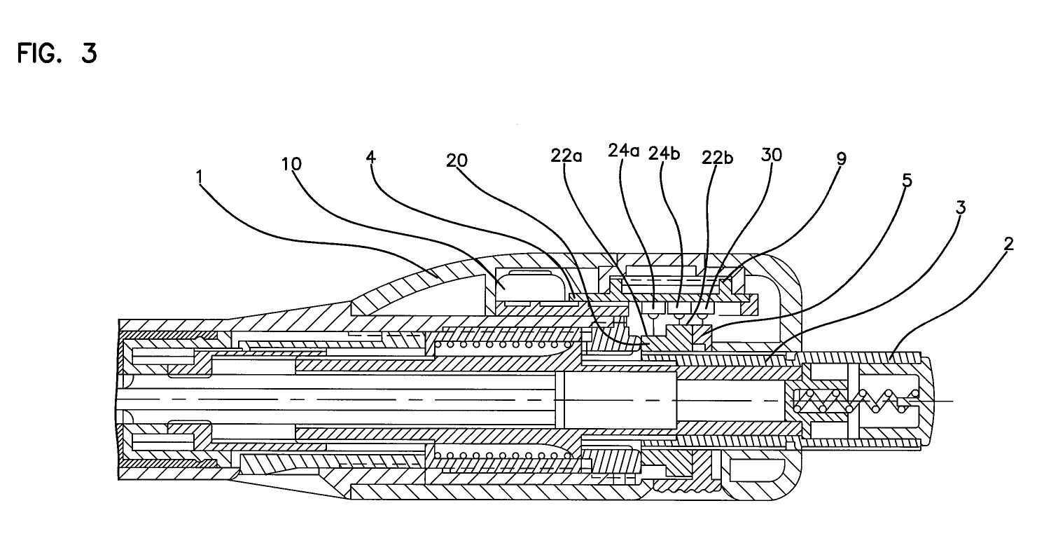

In accordance with the present invention, FIG. 3 illustrates an exemplary embodiment of an injection device with an exemplary embodiment of a dose setting module. The injection device has a housing 1, in which a dose setting mechanism and an administering mechanism of the injection device are accommodated. The dose setting mechanism has a dose setting knob 2, which projects out from the housing. 1. In the extension, the dose setting knob 2 has a sleeve 3 inside the housing 1, which transmits a rotating motion of the dose setting knob 2 to the dose setting mechanism in order to set a dose. Accordingly, the sleeve 3 moves inside the housing 1 about the longitudinal axis of the injection device and relative to the housing 1. The dose setting knob 2 can be pushed into the housing 1 to administer a product dose, as a result of which the sleeve 3 moves in the longitudinal direction of the longitudinal axis of the injection device and is moved in the longitudinal direction with respect to the housing 1. A product dose is administered from the injection device when the dose setting knob 2 is depressed. As illustrated in FIG. 1, the administering mechanism is fitted with various other elements, although these are not specifically indicated. To explain the present invention, it is necessary to describe how the setting of the sleeve 3 with respect to the housing 1 affects other elements which move relative to one another. Accordingly, within the meaning of the invention, the housing 1 may be regarded as a first element and the sleeve 3 as a second element.

Secured to the housing 1 is a beam or slim plate 4, which is part of or attached to the housing 1 and on which three sensors in the form of inductive sensors 24a, 24b and 30 of the type described in connection with FIG. 1 are mounted or carried. The inductive sensors are mounted adjacent to one another in the longitudinal direction of the injection device. Mounted on the sleeve 3 lying opposite the sensors 24a and 24b is a switching ring 20, the surface profile 10 of which has a first profiled region 22a and a second profiled region 22b. The profiled regions 22a and 22b have a periodic surface structure in the form of two different, alternating height levels. To this end, steps of equal length are disposed in the circumferential direction on a plate seated on the sleeve 3, which are repeated after a specific distance. The plate is coupled with the sleeve 3 as it moves in rotation but remains stationary when the sleeve 3 moves in the longitudinal direction. As may be seen from FIG. 3, the inductive sensor 24a is disposed opposite the first profiled region 22a so that the magnetic field 13 of the inductive sensor 24a changes as a function of the first profiled region 22a. Furthermore, the inductive sensor 24b is disposed opposite the second profiled region 22b so that the magnetic field 13 of the inductive sensor 24b changes as a function of the second profiled region 22b.

The measurement signals of the inductive sensors 24a, 24b and 30 are forwarded to a microprocessor 10 for processing, and the position of the sleeve 3 relative to the housing 1 is determined on the basis of the measured data and converted into a value for the dose setting or for administering, for example. The determined values are output on a display 9 disposed underneath a transparent region of the housing 1.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device comprising a dose setting module comprising at least one sensor element and at least one sensor actuating element, wherein the at least one sensor element and at least one sensor actuating element at least partially overlap along a longitudinal axis of the injection device and can be moved into at least one position in which they at least partially overlap circumferentially relative to the injection device, and wherein the at least one sensor element generates a magnetic field and, in the at least one position, the at least one sensor actuating element causes a change in the magnetic field.

2. The injection device according to claim 1, further comprising an evaluating unit configured so that when there is no change in a magnetic field of a sensor element the evaluating unit generates a low signal for said sensor element and when a change is detected in the magnetic field of a sensor element the evaluating unit generates a high signal for said sensor element.

3. The injection device according to claim 2, wherein at least one sensor element of the at least one sensor elements is a Hall-effect sensor.

4. The injection device according to claim 2, further comprising a resetting mechanism comprising at least one resetting element moveable into at least one position circumferentially relative to the injection device by turning or sliding, whereby the resetting element and at least one of the at least one sensor elements at least partially overlap circumferentially.

5. The injection device according to claim 2, further comprising a reference sensor for detecting an external magnetic field, said reference sensor not overlapping any sensor actuating element longitudinally relative to the injection device.

6. The injection device according to claim 5, wherein the dose setting module further comprises an electrically conductive housing shell able to screen the sensor elements and the sensor actuating elements from the external magnetic field.

7. A dose setting module for an injection device, said dose setting module comprising at least one sensor element for measuring a magnetic field and at least one sensor actuating element, the at least one sensor element and at least one sensor actuating element at least partially overlapping in the longitudinal direction of the injection device, wherein at least one of the at least one sensor elements and at least one of the at least one sensor actuating elements can be moved into at least one position in circumferentially relative to the injection device by turning or sliding, wherein the at least one sensor element and at least one sensor actuating element at least partially overlap circumferentially, and wherein the at least one sensor element is able to generate a magnetic field and the at least one sensor actuating element can cause a change in the magnetic field in said at least one position.

8. The dose setting module as claimed in claim 7, comprising from two to six sensor elements, the sensor elements offset from one another in the longitudinal direction of the injection device.

9. The dose setting module as claimed claim 8, comprising from two to six sensor elements, the sensor elements offset from one another in the circumferential direction of the injection device.

10. The dose setting module as claimed claim 7, comprising from two to six sensor elements, the sensor elements offset from one another in the circumferential direction of the injection device.

11. The dose setting module as claimed in claim 7, comprising from two to six sensor actuating elements, the sensor actuating elements offset from one another in the longitudinal direction of the injection device.

12. The dose setting module as claimed in claim 11, comprising two or more sensor actuating elements, wherein at least two of the sensor actuating elements are offset from one another in the circumferential direction of the injection device.

13. The dose setting module as claimed in claim 7, comprising two or more sensor actuating elements, wherein at least two of the sensor actuating elements are offset from one another in the circumferential direction of the injection device.

14. The dose setting module as claimed in claim 7, wherein at least one sensor element is a Hall-effect sensor configured to detect a change in the magnetic field.

15. The dose setting module as claimed in claim 7, further comprising an evaluating unit configured so that when there is no change in a magnetic field of a sensor element the evaluating unit emits a low signal for said sensor element and when a change is detected in the magnetic field of a sensor element the evaluating unit emits a high signal for said sensor element.

16. The dose setting module as claimed in claim 7, further comprising a reference sensor element for detecting an external magnetic field, wherein the reference sensor element does not overlap with any sensor actuating system in the longitudinal direction of the injection device.

17. The dose setting module as claimed in claim 7, wherein the at least one sensor element comprises a coil and a magnetic core and is designed so that it can generate one of a magnetic field or a concentric magnetic field.

18. The dose setting module as claimed in claim 7, wherein the at least one sensor element and the at least one sensor actuating element are separated from one another by a housing part.

19. The dose setting module as claimed in claim 7, further comprising an electrically conductive housing shell around the dose setting module so that the electrically conductive housing shell screens the at least one sensor element and the at least one sensor actuating element from an external magnetic field.

20. The dose setting module as claimed in claim 7, wherein at least two sensor actuating elements have different lengths.

21. The dose setting module as claimed in claim 7, further comprising a resetting mechanism at least partially overlapping a sensor element in the longitudinal direction of the injection device.

22. The dose setting module as claimed in claim 21, wherein the resetting mechanism comprises at least one resetting element moveable into at least one position in the circumferential direction of the injection device by turning or sliding, whereby the resetting element and a sensor element at least partially overlap in the circumferential direction of the injection device.

23. A method of detecting a dose setting in an infusion or injection device comprising at least one sensor element, at least one sensor actuating element and an evaluation unit, wherein a magnetic field is generated by the at least one sensor element, the method comprising the steps of moving the at least one sensor element and at least one sensor actuating element into a relative position by turning or sliding, in which position the at least one sensor element and at least one sensor actuating element at least partially overlap and the at least one sensor actuating element causes a change in the magnetic field of the at least on sensor element.

24. The method as claimed in claim 23, wherein the change in the magnetic field at a sensor element is detected by the sensor element at which the change in magnetic field occurred.

25. The method as claimed in claim 24, wherein the infusion or injection device further comprises a reference sensor and wherein an external magnetic field is detected by the reference sensor.

26. The method as claimed in claim 25, wherein the external magnetic field is detected when changes are detected in the magnetic field of the respective sensor element at all of the at least one sensor elements.

27. The method as claimed in claim 23, wherein the evaluation unit generates a first signal when there are changes in the magnetic field of the at least one sensor element and a second signal different from the first signal when there is no change in the magnetic field of the at least one sensor element.

28. The method as claimed in claim 25, wherein the evaluation unit regulates the magnetic field at the at least one sensor element on the basis of the detected external magnetic field so that the external magnetic field is compensated at the at least one sensor element.

29. The method as claimed in claim 28, wherein the evaluation unit generates an error signal if the detected external field exceeds a pre-set value at which the external field can no longer be compensated.

30. The method as claimed in claim 23, wherein when there is a drop below a pre-set critical value for the change in the magnetic field at the at least one sensor element, the evaluation unit generates a first signal and when the pre-set critical value for the change in the magnetic field at the at least one sensor element is exceeded the evaluation unit generates a second signal different from the first signal.
